SEQUENCE LISTING (2 sequences)

SEQ ID NO: 1 - DNA, 5641 bp, Homo sapiens
  CDS (117)..(4556)
cggaggaggacgcgagccccttgcgggcggtcatcacagcccagcctcggggctgccacagcgcgttgcgcctgtgcgccctcggtccccgcgtccactgagcgccgcgctcggggatggggcccggccggccggcccccgcgccctggcctcgtcacctgctgcgctgcgtcctgctcctcgggtgcctgcacctcggccgtcccggcgcccctggggacgccgccctcccggaacccaacgtcttcctcatcttcagccatggactgcagggctgcctggaggcccagggcgggcaggtcagagtcaccccggcttgcaataccagcctccctgcccagcgctggaagtgggtctcccgaaaccggctattcaacctgggtaccatgcagtgcctgggcacaggctggccaggcaccaacaccacggcctccctgggcatgtatgagtgtgaccgggaagcactgaatcttcgctggcattgtcgtacactgggtgaccagctgtccttgctcctgggggcccgcaccagcaacatatccaagcctggcacccttgagcgtggtgaccagacccgcagtggccagtggcgcatctacggcagcgaggaggacctatgtgctctgccctaccacgaggtctacaccatccagggaaactcccacggaaagccgtgcaccatccccttcaaatatgacaaccagtggttccacggctgcaccagcacgggccgcgaggatggtcacctgtggtgtgccaccacccaggactacggcaaagacgagcgctggggcttctgccccatcaagagtaacgactgcgagaccttctgggacaaggaccagctgactgacagctgctaccagtttaacttccagtccacgctgtcgtggagggaggcctgggccagctgcgagcagcagggtgcggatctgctgagcatcacggagatccacgagcagacctacatcaacggcctcctcactgggtacagctccaccctgtggatcggcttgaatgacttggacacgagcggaggctggcagtggtcggacaactcgcccctcaagtacctcaactgggagagtgaccagccggacaaccccagtgaggagaactgtggagtgatccgcactgagtcctcgggcggctggcagaaccgtgactgcagcatcgcgctgccctatgtgtgcaagaagaagcccaacgccacggccgagcccacccctccagacaggtgggccaatgtgaaggtggagtgcgagccgagctggcagcccttccagggccactgctaccgcctgcaggccgagaagcgcagctggcaggagtccaagaaggcatgtctacggggcggtggcgacctggtcagcatccacagcatggcggagctggaattcatcaccaagcagatcaagcaagaggtggaggagctgtggatcggcctcaacgatttgaaactgcagatgaattttgagtggtctgacgggagccttgtgagcttcacccactggcacccctttgagcccaacaacttccgggacagtctggaggactgtgtcaccatctggggcccggaaggccgctggaacgacagtccctgtaaccagtccttgccatccatctgcaagaaggcaggccagctgagccagggggccgccgaggaggaccatggctgccggaagggttggacgtggcacagcccatcctgctactggctgggagaagaccaagtgacctacagtgaggcccggcgcctgtgcactgaccatggctctcagctggtcaccatcaccaacaggttcgagcaggccttcgtcagcagcctcatctacaactgggagggcgagtacttctggacggccctgcaggacctcaacagcaccggctccttcttctggctcagtggggatgaagtcatgtacacccactggaaccgggaccagcccgggtacagccgtgggggctgcgtggcgctggccactggcagcgccatggggctgtgggaggtgaagaactgtacctcgttccgggcccgctacatctgccggcagagcctgggcactccagtgacgccggagctgccggggccagatcccacgcccagcctcactggctcctgtccccagggctgggcctcggacaccaaactccggtattgctataaggtgttcagctcagagcggctgcaggacaagaagagctgggtccaggcccagggggcctgccaggagctgggggcccagctgctgagcctggccagctacgaggaggagcactttgtggccaacatgctcaacaagatcttcggtgaatcagaacccgagatccacgagcagcactggttctggatcggcctgaaccgtcgggatcccagagggggtcagagttggcgctggagcgacggcgtagggttctcttaccacaatttcgaccggagccggcacgacgacgacgacatccgaggctgtgcggtgctggacctggcctccctgcagtgggtggccatgcagtgcgacacacagctggactggatctgcaagatccccagaggtacggacgtgcgggagcccgacgacagccctcaaggccgacgggaatggctgcgcttccaggaggccgagtacaagttctttgagcaccactccacgtgggcgcaggcgcagcgcatctgcacgtggttccaggccgagctgacctccgtgcacagccaggcagagctagacttcctgagccacaacttgcagaagttctcccgggcccaggagcagcactggtggatcggcctgcacacctctgagagcgatgggcgcttcagatggacagatggttccattataaacttcatctcctgggcaccaggcaaacctcggcctgtcggcaaggacaagaagtgcgtgtacatgacagccagccgagaggactggggggaccagaggtgcctgacagccttgccctacatctgcaagcgcagcaacgtcaccaaagaaacgcagcccccagacctgccaactacagccctggggggctgcccctctgactggatccagttcctcaacaagtgttttcaggtccagggccaggaaccccagagccgggtgaagtggtcagaggcacagttctcctgtgaacagcaagaggcccagctggtcaccatcacaaaccccttagagcaagcattcatcacagccagcctgcccaatgtgacctttgacctttggattggcctccatgcctcgcagagggacttccagtgggtggagcaggagcctttgatgtatgccaactgggcacctggggagccctctggccctagccctgctcccagtggcaacaaaccgaccagctgtgcggtggtcctgcacagcccctcagcccacttcactggccgctgggacgatcggagctgcacggaggagacccatggcttcatctgccagaagggcacggacccctccctgagcccgtccccagcagcgctgccccccgccccgggcactgagctctcctacctcaacggcaccttccggctgcttcagaagccgctgcgctggcacgatgccctcctgctgtgtgagagccacaatgccagcctggcctacgtgcccgacccctacacccaggccttcctcacgcaggctgcccgagggctgcgcacgccgctctggattgggctggctggcgaggagggctctcggcggtactcctgggtctcagaggagccgctgaactacgtgggctggcaggacggggagccgcagcagccggggggctgtacctacgtagatgtggacggggcctggcgcaccaccagctgtgacaccaagctgcagggggctgtgtgtggggttagcagtgggccccctcctccccgaagaataagctaccatggcagctgtccccagggactggcagactccgcgtggattcccttccgggagcactgctattctttccacatggagctgctgctgggccacaaggaggcgcgacagcgctgccagagagcgggtggggccgtcctgtctatcctggatgagatggagaatgtgtttgtctgggagcacctgcagagctatgagggccagagtcggggcgcctggctgggcatgaacttcaaccccaaaggaggcactctggtctggcaggacaacacagctgtgaactactccaactgggggcccccgggcttgggccccagcatgctgagccacaacagctgctactggattcagagcaacagcgggctatggcgccccggcgcttgcaccaacatcaccatgggtgtcgtctgcaagcttcctcgtgctgagcagagcagcttctccccatcagcgcttccagagaacccagcggccctggtggtggtgctgatggcggtgctgctgctcctggccttgctgaccgcagccctcatcctttaccggaggcgccagagcatcgagcgcggggcctttgagggtgcccgctacagccgcagcagctccagccccaccgaggccactgagaagaacatcctggtgtcagacatggaaatgaatgagcaacaagaatagagccaggcgcgtgggcagggccagggcgggaggagctggggagctggggccctgggtcagtctggccccccaccagctgcctgtccagttggcctatggaagggtgcccttgggagtcgctgttgggagccggagctgggcagagcctgggctggtggggtgccaccctcccacaagggctgggctgagacccagctgagtgcagcgtggcgtttccctttctgggggggcctgaggtcttgtcacctggtcctgtgcccccacaggaaccagaggtaggatgggagggggaacgagagcctctttctccccagagcccccggcccaggcctgttgatccgcgccccaggacccccttctttgcagagcccgaggagcctcccctgtcccctcgggcagatctgttgtgtctctcttcccacctggcagcctcagctctgtgcccctcaccctgctccctctcgccccttctctcccaccccttccttctgagccgggccctggggattggggagccctcttgttcctgatgagggtcagctgagggggctgagcatccatcactcctgtgcctgctggggtggctgtggggcgtggcaggaggggcctaggtgggttgggcctgagaaccagggcacgggtgtggtgtctgctgggctggagataagactggggagagacaccccaacctcccagggtgggagctgggccgggctgggatgtcatctcctgccgggcgggggagggctctgcccctggaagagtcccctgtggggaccaaaataagttccctaacatctccagctcctggctctggtttggagcaaggggaagggttgccagagtcctgggggccccagaggagcaggagtctgggagggcccagagttcaccctctagtggatccaggaggagcagcacccgagccctggagtggcccagtacccttccaagaggccacagtcccagccaggacaaagtatgcggcccatcctggtgcgacagcgtgggacaatgtgaacatggactcgaagacatggccctttctctgtagttgattttttaaatgtgccattattgttttt

SEQ ID NO: 2 - protein, 1479 aa, Homo sapiens
MGPGRPAPAPWPRHLLRCVLLLGCLHLGRPGAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRKGWTWHSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYICRQSLGTPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRGTDVREPDDSPQGRREWLRFQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICKRSNVTKETQPPDLPTTALGGCPSDWIQFLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFICQKGTDPSLSPSPAALPPAPGTELSYLNGTFRLLQKPLRWHDALLLCESHNASLAYVPDPYTQAFLTQAARGLRTPLWIGLAGEEGSRRYSWVSEEPLNYVGWQDGEPQQPGGCTYVDVDGAWRTTSCDTKLQGAVCGVSSGPPPPRRISYHGSCPQGLADSAWIPFREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVCKLPRAEQSSFSPSALPENPAALVVVLMAVLLLLALLTAALILYRRRQSIERGAFEGARYSRSSSSPTEATEKNILVSDMEMNEQQE

(19) United States
(12) Patent Application Publication
(10) Pub. No.: US 2007/0072244 A1
(43) Pub. Date: Mar. 29, 2007

(54) Use of the endo-180 gene and polypeptide for diagnosis and treatment

(75) Inventors: Orna Mor, Kiryat Ono (IL); Elena Feinstein, Rehovot (IL); Alexander Faerman, Gan Yavne (IL)

Correspondence Address: COOPER & DUNHAM, LLP, 1185 AVENUE OF THE AMERICAS, NEW YORK, NY, 10036, US

(73) Assignee: QUARK BIOTECH, INC., Fremont, CA (US)

(21) Appl. No.: 10/557,089

(22) PCT Filed:
(86) PCT No.: PCT/IL04/00423

Related U.S. Application Data
(60) Provisional application No. 60/472,102, filed on May 19, 2003.

(51) Int. Cl. G01N 33/567; A61K 38/17

(52) U.S. Cl. 435/7.2; 514/12

(57) ABSTRACT

This application is directed to a process of identifying a compound capable of modulating activity of a human ENDO180 receptor that comprises the steps of measuring the binding of the ENDO180 receptor to an interactor with which the ENDO180 receptor interacts specifically in vivo, in the absence or presence of a compound, and determining whether the binding of the ENDO180 receptor to the interactor is affected by the compound. This application is also directed to use of a compound identified by that process in the preparation of a medicament for therapy of disease, in particular fibrosis. This application also relates to the use of ENDO180 modulators in treatment of disease.

### FIELD OF THE INVENTION

The present invention relates to the identification and isolation of polynucleotide sequences, the expression of which is changed in various pathologies, and use of these isolated polynucleotides as probes for diagnosis, for screening of treatment modalities and as targets for modulation in fibrosis in general, for chronic renal insufficiency and for kidney fibrosis and glomerulosclerosis, in particular.

### BACKGROUND OF THE INVENTION

Fibrotic Diseases

Fibrotic diseases are all characterized by the excess production of a fibrous material called the extracellular matrix, which contributes to abnormal changes in tissue architecture and interferes with normal organ function. Millions of people world—wide suffer from these chronic diseases, that are often life threatening. Unfortunately, although fibrosis is widely prevalent, debilitating and often life threatening, there is no effective treatment currently available.

The human body responds to trauma and injury by scarring. Fibrosis, a type of disorder characterized by excessive scarring, occurs when the normal wound healing response is disturbed. During fibrosis, the wound healing response continues causing an excessive production and deposition of collagen.

Although fibrotic disorders can be acute or chronic, the disorders share a common characteristic of excessive collagen accumulation and an associated loss of function when normal tissue is replaced with scar tissue.

Fibrosis results from diverse causes, and may be established in various organs. Cirrhosis, pulmonary fibrosis, sarcoidosis, keloids, hypertension and kidney fibrosis, are all chronic diseases that induce a progressive fibrosis which causing a continuous loss of tissue function.

Acute fibrosis (usually with a sudden and severe onset and of short duration) occurs as a common response to various forms of trauma including accidental injuries (particularly injuries to the spine and central nervous system), infections, surgery (cardiac scarring following heart attack), burns, environmental pollutants, alcohol and other types of toxins, acute respiratory distress syndrome, radiation and chemotherapy treatments. All tissues damaged by trauma are prone to scar and become fibrotic, particularly if the damage is repeated. Deep organ fibrosis is often extremely serious because the progressive loss of organ function leads to morbidity, hospitalization, dialysis, disability and even death. Fibrotic diseases or diseases in which fibrosis is evident include pulmonary fibrosis, interstitial lung disease, human fibrotic lung disease, liver fibrosis, cardiac fibrosis, macular degeneration, retinal and vitreal retinopathy, myocardial fibrosis, Grave's ophthalmopathy, drug induced ergotism, cardiovascular disease, atherosclerosis/restenosis, keloids and hypertrophic scars, cancer, Alzheimer's disease, scarring, scleroderma, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease and inflammatory bowel disease, including collagenous colitis.

For further information on different types of fibrosis see: Molina V, Blank M, Shoenfeld Y. (2002), “Fibrotic diseases”, Harefuah, 141(11): 973-8, 1009; Yu L, Noble N A, Border W A (2002), “Therapeutic strategies to halt renal fibrosis”, Curr Opin Pharmacol. 2(2):177-81; Keane W F, Lyle P A. (2003), “Recent advances in management of type 2 diabetes and nephropathy: lessons from the RENAAL study”, Am J Kidney Dis. 41(3 Suppl 2): S22-5; Bohle A, Kressel G, Muller C A, Muller G A. (1989), “The pathogenesis of chronic renal failure”, Pathol Res Pract. 185(4):421-40; Kikkawa R, Togawa M, Isono M, Isshiki K, Haneda M. (1997), “Mechanism of the progression of diabetic nephropathy to renal failure”, Kidnzey Int Suppl. 62:S39-40; Bataller R, Brenner D A. (2001), “Hepatic stellate cells as a target for the treatment of liver fibrosis”, Semin Liver Dis. 21(3):437-51; Gross T J, Hunninghake G W, (2001) “Idiopathic pulmonary fibrosis”, N Engl J Med. 345(7):517-25; Frohlich E D. (2001) “Fibrosis and ischemia: the real risks in hypertensive heart disease”, Am J Hypertens;14(6 Pt 2):194S-199S.

Diabetic Nephropathy

Diabetic nephropathy, hallmarks of which are glomerulosclerosis and kidney fibrosis, is the single most prevalent cause of end-stage renal disease in the modern world, and diabetic patients constitute the largest population on dialysis. Such therapy is costly and far from optimal. Transplantation offers a better outcome but suffers from a severe shortage of donors. More targeted therapies against diabetic nephropathy (as well as against other types of kidney pathologies) are not developed, since molecular mechanisms underlying these pathologies are largely unknown. Identification of an essential functional target gene that is modulated in the disease and affects the severity of the outcome of diabetes nephropathy has a high diagnostic as well as therapeutic value.

It is known in the art that many pathological processes in the kidney eventually culminate in similar or identical morphological changes, namely glomerulosclerosis and fibrosis. Human kidney disease may evolve from various origins including glomerular nephritis, nephritis associated with systemic lupus, cancer, physical obstructions, toxins, metabolic disease and immunological diseases, all of which culminate in kidney fibrosis. The meaning of this phenomenon is that different types of insults converge on the same single genetic program resulting in two hallmarks of fibrosis: the proliferation of fibroblasts and overproduction by them of various protein components of connective tissue. In addition, thickening of the basal membrane in the glomeruli accompanies interstitial fibrosis and culminates in glomerulosclerosis. Genes encoding proteins that are involved in kidney fibrosis and glomerulosclerosis may be roughly divided into two groups:
1. Genes, the expression of which leads to the triggering of proliferation of fibroblasts and overproduction by them of various protein components of connective tissue. These may be specific to different pathological conditions;
2. Genes, the expression of which leads to the execution of the “fibrotic or sclerotic programs”. These may be common to all renal pathologies leading to fibrosis and glomerulosclerosis.

The identification of genes that belong to the second group should contribute to the understanding of molecular mechanisms that accompany fibroblast and mesangial cell proliferation and hypersecretion, and may constitute genetic targets for drug development, aimed at preventing renal failure. Application of such drugs is expected to suppress, retard, prevent, inhibit or attenuate progression of fibrosis and glomerulosclerosis.

A useful way to assess the development of renal diseases involving fibrosis and glomerulosclerosis is to characterize gene expression in established animal models of kidney diseases. Examples of such models include without limitation: (i) fa/fa rats—animals genetically deficient in leptin receptor that develop insulin resistant diabetes (type II diabetes) with progressive diabetic nephropathy, and (ii) GK rats—which are genetically manipulated, NIDDM phenotype rats. Another animal model in which mainly kidney fibrosis is evident, but without a background of diabetes, is unilateral ureteral obstruction (UUO) in which interstitial fibrosis is rapid and occurs within days following the obstruction. 5/6 nephrectomy is another useful animal model for chronic renal insufficiency (CRI) in which fibrosis is evident.

Additional aspects of research may be based on an in vitro model system involving culture of human fibroblasts in vitro under conditions mimicking various parameters of the cell microenvironment existing in CRI and fibrosis. These include treatment with high concentrations of glucose (modeling hyperglycemia), low concentrations of glucose, hypoxia (both modeling ischemic conditions that develop in the kidney following fibrosis and glomerulosclerosis), and TGF-β—one of the recognized pathogenic factors in fibrosis. Such in vitro model systems may complement the animal models in several important aspects: First, the system is fibroblast-specific; accordingly, none of the interferences often found in complex tissues that contain many cell types are present. Second, the cells are of human origin, unlike the animal models. Furthermore, the insults are specific and of various concentrations and duration, thus enabling the investigation of both acute and chronic responses.

ENDO180/uPARAP

ENDO180, known also as uPARAP (urokinase plasminogen activator receptor associated protein), is an internalization receptor that directs its bound ligands to degradation in the endosomes. It is part of a triple complex with urokinase type plasmin activator (uPA) and urokinase-type plasmin activator receptor (uPAR), thus being involved in the production of plasmin from plasminogen. Plasmin, in turn, is known to play a role in both ECM turnover and proteolytic conversion of latent TGF-beta into its active form.

Besides being involved in the production of plasmin, the triple complex is known to be involved in the activation of MMP proenzymes, to function against fibrin to bind several collagens and to be involved in general turnover of extracellular matrix. This complex also takes part in cell adhesion and signal transduction (Bherendt el al, JBC275: 1993-2002, 2000).

It is therefore suggested that END0180 is a multifunctional receptor that displays Ca²⁺ dependent lectin activity, and participates in extracellular matrix degradation and remodeling (Chambers et al, 2001, 47^{th} Annual Meeting, Orthopaedic Research Society, Feb. 25-28, 2001, San Francisco, Calif.).

ENDO180 is localized on the cell surface, in clathrin coated pits (Isacke et al., 1990 Mol. Cell. Biol. 10: 2606-2618; Sheikh et al., 2000, J. Cell. Sci. 113: 1021-1032) and in endosomes. It is mainly expressed in fibroblasts, endothelial cells and macrophages. In situ hybridization showed its expression in highly vascularized organs. It has also been found in bone-forming regions in mouse embryos (Wu et al., 1996, J. Biol. Chem. 271:21323-21330), and in osteoblasts and osteocytes at sites of endochondral and intramembranous ossification during development (Engelhohn et al., 2001b, Trends Cardiovasc. Med. 11: 7-13).

Recently it was shown that Endo180 interacts with various types of collagen (collagen IV and collagen V) and is therefore considered crucially engaged in matrix degradation (Niels B. Biol Chem. February 2004;385(2):103-36); Kjoller L et al, Exp Cell Res. 2004 Feb. 1;293(1):106-16

Structural Information

Nucleotide Sequence: Human—5641 bp NM_{—}006039; ORF—4439bp (117-4441bp);
Mouse—5818 bp (Acc: MMU56734); ORF—1479aa, 167kda.

Protein sequence: NP_{—}006030_endo 180.fcgi 1479 aa (Behrendt 2000, acc: NP_{—}006030).

Domains (human protein NP_{—}006030 by smart)—type 1 membrane protein with:
1-31aa SP (signal peptide)
41-161aa cysteine rich N-terminal domain
180-228 aa FNII (fibronectin type II) domain
8 CDR (carbohydrate recognition domain):
   235-360 aa 1CRD
   382-505 aa 2CRD
   521-645 aa 3CRD
   669-809 aa 4CRD
   825-951 aa 5CRD
   972-1108 aa 6CRD
   1161-1244 aa 7CRD
   1261-1394 aa 8CRD
1413-1435 aa 1 TM (transmembrane domain)
1437-1479 aa-cytoplasmic domain

Similarity between species: 87% homology between mouse and human —on nucleic acid level (Sheikh et al., 2000, J Cell. Sci. 113: 1021-1032).

Motifs: Putative endocytosis motives identical in mouse and human ENDO180. The location below refers to the human ORF:
1. Tyr 1452 (FxNxxY)
2. Leu 1468/Val1469 (E-LV)—the amino acids recited are for the human ENDO180.

ENDO180 shares homology with the macrophage mannose receptor family: mannose receptor, phosphlipase A₂ and DEC-205/MR6 (Isacke et al., 1990 Mol. Cell. Biol. 10: 2606-2618; Sheikh et al., 2000, J. Cell. Sci. 113: 1021-1032; Behrendt et al., 2000, J. Biol. Chem. 275: 1993-2002). This family grouping is based on an overall structural conservation with the four receptors containing a large extracellular domain comprising an N-terminal signal sequence followed by a cysteine-rich domain, a fibronectin type II domain (FNII), and 8 or 10 C-type lectin-like domains (CTLDs). As a family, these receptors have two striking features: First, although they belong to the large C-type lectin superfamily, they uniquely contain multiple CTLDs within a single polypeptide backbone (Taylor M. E., 1997 Glycobiology 7: v-vii; McKay et al, 1998, Eur. J. Immunol. 28: 4071-4083; Howard M. J. and Isacke C. M., 2002 JBC Papers in Press. Published on Jun. 14, 2002 as Manuscript M203631200). Second, they share the ability to be recycled between the plasma membrane and intercellular compartments of the cell (Isacke et al, 1990, Mol. Cell. Biol. 1-0: 2606-2618; Zvaritch et al., 1996, J. Biol. Chem. 271: 250-257).

ENDO180 was also shown to internalize antibodies directed against it (Isacke, 1990, Mol. Cell. Biol. 10: 2606-2618), a feature common to all mannose family proteins that function as internalization receptors, and constitutively internalized from the plasma membrane via clathrin-mediated endocytosis and recycled back to the cell surface (Howard M. J. and Isacke C. M., 2002 JBC Papers in Press. Published on Jun. 14, 2002 as Manuscript M203631200).

Engelhohm et al, 2003, The Jornal of Cell Biology 160:1009-1015, recently reported that ENDO180 is essential for the uptake of collagen by fibroblasts.

Zhang et al., 2003, J. Am. Soc. Nephrol. 14(5): 1254-1271, has reported that renal uPAR attenuates the fibrogenic response to renal injury.

The following patent publications also relate to the ENDO180 receptor: U.S. Pat. No. 6,117,977; WO 97/40154 and WO 00/58473.

### SUMMARY OF THE INVENTION

The main object of the present invention is the identification and isolation of novel genetic targets that may be used for development of drugs to treat many diseases such as tissue fibrosis in general, chronic renal insufficiency (CRI), chronic renal failure (CRF), and kidney fibrosis and glomerulosclerosis, in particular, as well as osteoporosis and osteoarthritis. A further object of the present invention is the identification and isolation of novel genetic targets that may be used for development of diagnostic and prognostic applications. It is further an object of the present invention to identify and isolate novel genetic targets for development of drugs to treat chronic renal insufficiency and other renal diseases, such as diabetic nephropathy, and usage of such targets as a tool for diagnostic and prognostic applications. It is yet a further object of the present invention to identify and isolate novel genetic targets for development of drugs to treat the hallmarks of diabetic nephropathy, namely glomerulosclerosis and renal fibrosis.

The present invention provides novel targets for development of novel therapeutic and diagnostic means, via large-scale microarray-based analysis of gene expression in nephropathy and more specifically in diabetic nephropathy and kidney fibrosis models in vivo and in vitro. In one embodiment, the present invention identifies up—or down—regulator (responder) genes for gene therapy, diagnostics and therapeutics that have direct causal relationships between a fibrotic nephropathological disease and its related pathologies. More preferably, the present invention identifies the ENDO180 gene as a modulator gene in the above-mentioned models.

The present invention further provides a process referred to herein as a screening assay for identifying modulators, i.e., candidate or compounds or agents including but not limiting to neutralizing antibodies, peptides, peptido-mimetics, small molecules and other drugs, which bind to ENDO180 or have an effect on ENDO180 expression or on ENDO180 activity.

The compound or agent discovered by the above-mentioned screening assay that may affect signaling via the ENDO180 receptor can be used in kidney fibrosis to modulate collagen uptake, fibronectin and/or MMP uptake, fibroblast adhesion and migration on fibrillar collagen matrices, mesangial cell proliferation. It can further be used to slow the pace of or inhibit glomerulosclerosis, to reduce the proliferation of fibroblasts, to inhibit the accumulation of extracellular matrix and to reduce or limit the formation of fibrotic regions in the kidney.

### BRIEF DESCRIPTION OF THE FIGURES

FIG. 1. This figure represents the nucleic acid sequence of the human ENDO180 gene (ACCESSION NM_{—}006039)—SEQ ID NO:1.

FIG. 2. This figure represents the amino acid sequence of the human ENDO180 gene (ACCESSION NP_{—}006030)—SEQ ID NO:2.

FIG. 3. This figure represents in vitro analysis of ENDO180 over-expression on growth rate and collagen accumulation in the ECM of rat1 fibroblasts.

FIG. 4. This figure, represents a construct used for the creation of transgenic mice expressing the FNII domain of ENDO180.

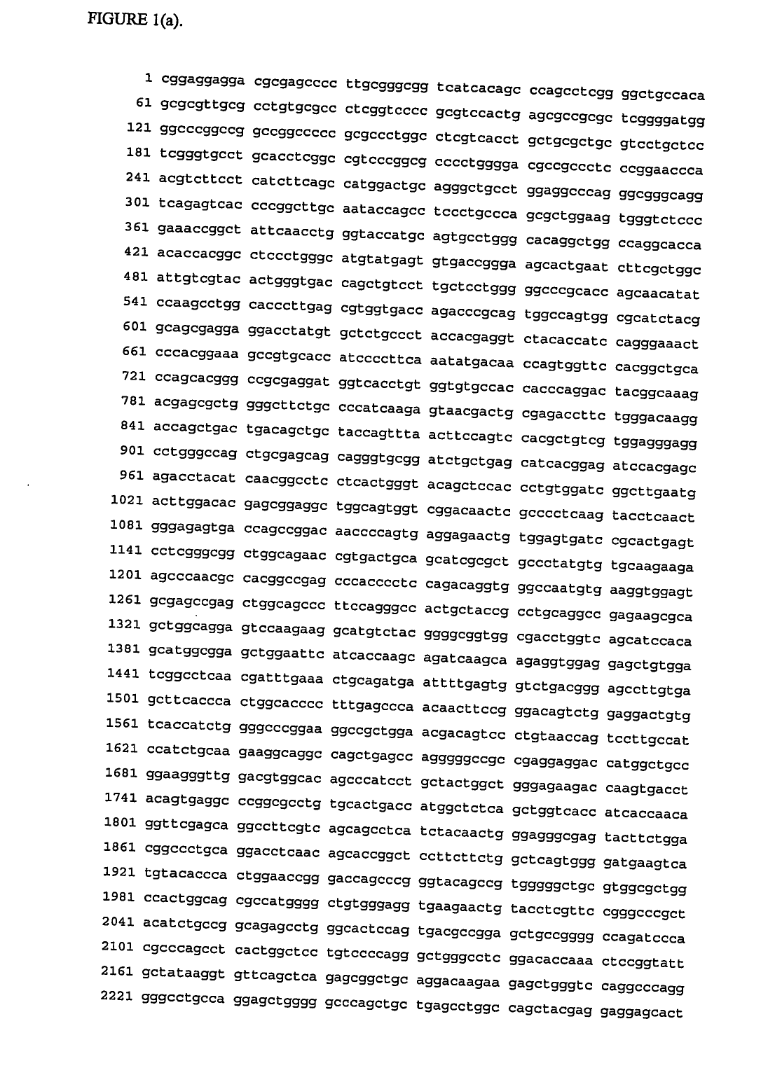

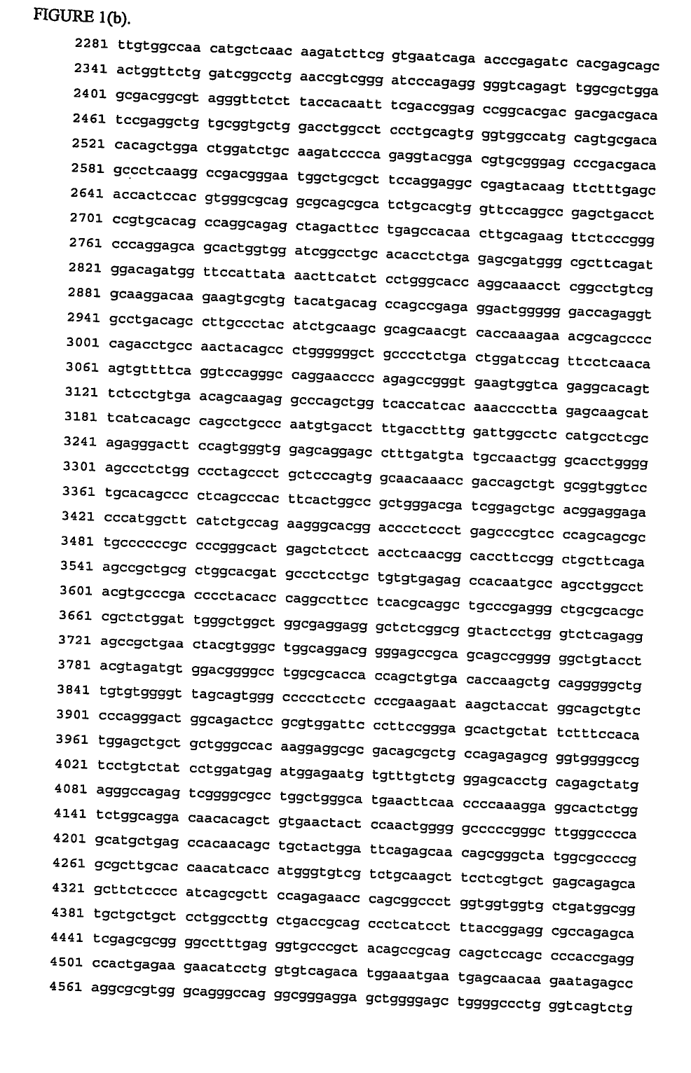

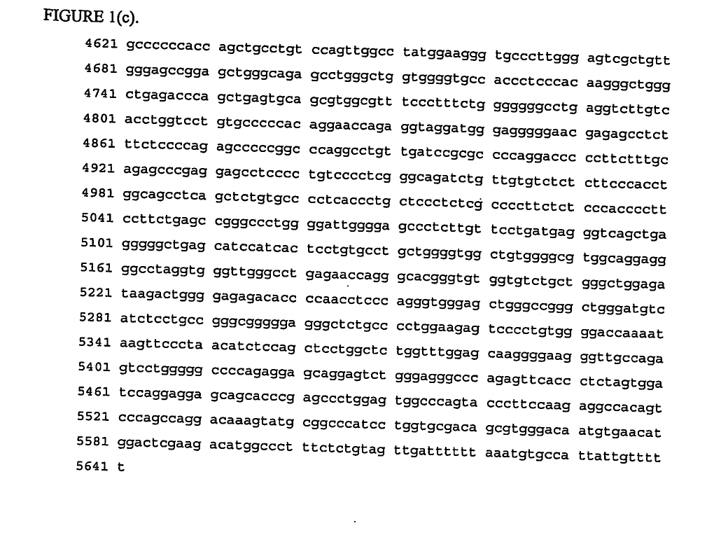

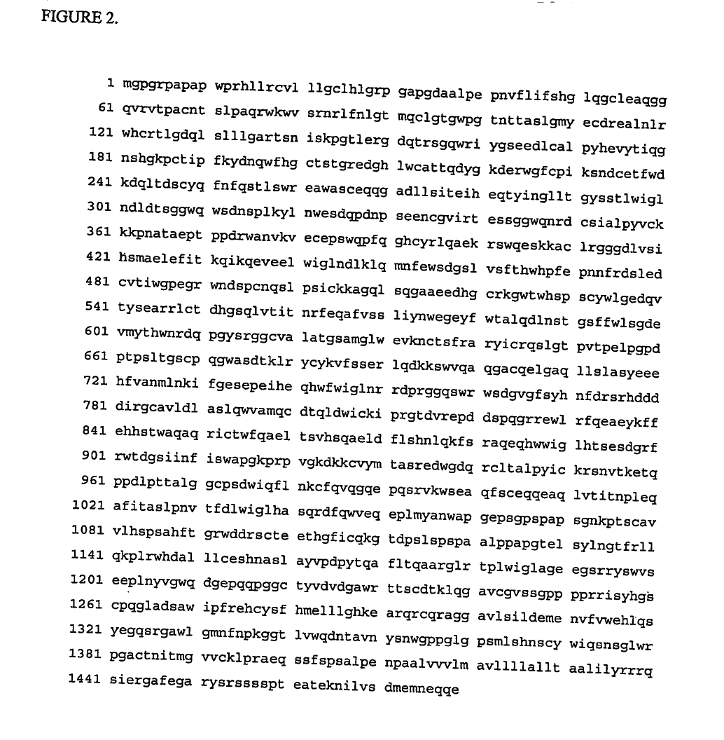

### DETAILED DESCRIPTION OF THE INVENTION

According to the present invention, purified, isolated and cloned nucleic acid sequences, specifically the nucleic acid sequence that encodes the ENDO180 receptor, associated with chronic renal failure, more specifically with nephropathy and even more specifically with diabetic nephropathy and with fibrotic and glomerulosclerotic kidneys and having sequences as specified herein or having complementary or allelic sequence variations thereto, are disclosed. Furthermore, a purified, isolated and cloned nucleic acid associated with nephropathy and having a sequence of SEQ ID NO:1 which encodes SEQ D NO:2 herein is also disclosed.

As used herein, the term “ENDO180 gene” is defined as any homolog of the ENDO180 gene having preferably 90% homology, more preferably 95% homology, and even more preferably 98% homology to the amino acid encoding region of SEQ ID NO:1 or nucleic acid sequences which bind to the ENDO180 gene under conditions of highly stringent hybridization, which are well-known in the art (for example, see Ausubel et al., Current Protocols in Molecular Biology, John Wiley and Sons, Baltimore, Md. (1988), updated in 1995 and 1998).

As used herein, the term “ENDO180” or “ENDO180 polypeptide” or “ENDO180 receptor” is defined as any homolog of the ENDO180 polypeptide having preferably 90% homology, more preferably 95% homology, and even more preferably 98% homology to SEQ ID NO:2, as either full-length or a fragments or a domain thereof, as a mutant or the polypeptide encoded by a spliced variant nucleic acid sequence, as a chimera with other polypeptides, provided that any of the above has the same or substantially the same biological function as the ENDO180 receptor. ENDO180 polypeptide, or an ENDO180 polypeptide homolog, may be present in different forms, including but not limited to soluble protein, membrane-bound (either in purified membrane preparations or on a cell surface), bead-bound, or any other form presenting ENDO180 protein or fragments and polypeptides derived thereof

Where the sequences are partial sequences, they may be used as markers/probes for genes that are modulated in fibrosis. In general these partial sequences which are designated “Expressed Sequence Tags” (ESTs), are markers for the genes actually expressed iii vivo. Generally, ESTs comprise DNA sequences corresponding to a portion of nuclear encoded MnRNA. The EST has a length that allows for polymerase chain reaction (PCR), and is used as a hybridization probe, with a unique designation for the gene with which it hybridizes (generally under conditions sufficiently stringent to require at least 95% base pairing). For a detailed description and review of ESTs and their functional utility see WO 93/00353 which is incorporated herein in its entirety by reference. WO 93/00353 further describes how the EST sequences can be used to identify the transcribed genes.

As used herein, an “interactor” is a molecule with which ENDO180 or an ENDO180 gene family member binds or interacts or activates in nature; for example, a molecule on the surface of a cell that expresses ENDO180 receptor, a molecule on the surface of a second cell, a molecule associated with the internal surface of a cell membrane or a cytoplasmic molecule. An interactor may be a ligand that has been internalized from extracellular milieu by ENDO180 receptor, for release in an endosomal compartment. An interactor may further be a ligand that is activated by ENDO180 alone or by ENDO180 as part of a complex with other components. An interactor may be a component of a signal transduction pathway that facilitates transduction of an extracellular signal from ENDO180 through the cell membrane and into the cell. An interactor, for example, can be a second intercellular protein that mediates downstream signaling from ENDO180. Specific interactors may be collagen and fibronectin.

As used herein, the term “compound” or “modulator” is defined as comprising any small chemical molecule, antibodies, neutralizing antibodies, antisense DNA or RNA molecules, siRNA, proteins, polypeptides and peptides including peptido-mimetics and dominant negatives, and expression vectors.

In one embodiment, the invention provides assays for screening candidates or compounds or modulators that bind to, modulate the activity of, or modulate the expression level of ENDO180. The compounds of the present invention can be obtained by using any of the numerous approaches in combinatorial and non-combinatorial library methods known in the art, including biological libraries (proteins, peptides, etc.), spatially addressable parallel solid phase or solution phase libraries, synthetic library methods, and natural product libraries.

The modulator of ENDO180 expression (transcription or translation) or polypeptide activity may be inter alia a small chemical molecule which generally has a molecular weight of less than 2000 daltons, more preferably less than 1000 daltons, even more preferably less than 500 daltons. Other modulators may be antibodies preferably neutralizing antibodies or fragments thereof including single chain antibodies, antisense oligonucleotides, antisense DNA or RNA molecules, siRNA, proteins, polypeptides and peptides including peptido-mimetics and dominant negatives, and expression vectors. These modulators may act as follows: small molecules may affect expression and/or activity; antibodies—may affect activity; all kinds of antisense—may affect ENDO180 expression; dominant negative polypeptides and peptidomimetics—may affect activity; expression vectors may be used inter alia for delivery of antisense or dominant-negative polypeptides.

Approaches have recently been developed that utilize small molecules, which can bind directly to proteins and can be used to alter protein function (for review see B. R. Stockwell, (2000) Nature Reviews/Genetics, 1, 116-125). As mentioned above, low molecular weight organic compounds can permeate the plasma membrane of target cells relatively easily and, therefore, methods have been developed for their synthesis. These syntheses, in turn, have yielded libraries that contain ligands for many proteins. Recent developments have brought a greatly increased variety of creatively selected, novel, small organic molecules that will function as powerful tools for perturbing biological systems. Such small molecules can be used to activate or inactivate specific members of a protein family.

Examples of methods for the synthesis of molecular libraries can be found in the art, for example, in DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carrell et al. (1994) Angew. Chem. Int. Ed. Engl. 33:2061; and in Gallop et al. (1994) J. Med. Chem. 37:1233.

In another aspect of the invention, ENDO180 protein can be used as “bait protein” in a two-hybrid assay or three-hybrid assay (e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins which bind to or interact with ENDO180 (“ENDO180-binding proteins”) and modulate ENDO180 activity. Such ENDO180-binding proteins are also likely to be involved in the propagation of signals by ENDO180 as, for example, upstream or downstream elements of the ENDO180 signaling pathway.

The present invention further provides a process for identifying a compound capable of modulating activity of a human ENDO1 80 receptor that comprises the steps of:
(i) measuring the binding of the ENDO180 receptor to an interactor with which the ENDO180 receptor interacts specifically in vivo, in the absence or presence of a compound; and
(ii) determining whether the binding of the ENDO180 receptor to said interactor is affected by said compound.

In accordance with the present invention, the ENDO180 receptor comprises consecutive amino acids, the sequence of which is set forth in SEQ ID NO: 2.

The chronic renal failure or the chronic renal insufficiency in the present invention may for example comprise kidney fibrosis or glomerulosclerosis or diabetic nephropathy, and the interactor in the above process may be, for example, collagen, fibronectin proliferation of cells, hypertrophy of tissue, endocytosis of extracellular components.

The present invention further provides a process for identifying a compound capable of modulating activity of a human ENDO180 receptor that comprises the steps of:
(i) measuring the binding of the ENDO180 receptor to an interactor with which the ENDO180 receptor interacts specifically in vivo;
(ii) contacting the ENDO180 receptor or an interactor with said compound; and
(iii) determining whether the binding of the ENDO180 receptor to said interactor is affected-by said compound.

In one embodiment of the invention, the measurement of step (i) in said processes of identifying a compound capable of modulating the activity of the ENDO180 receptor, is one of the multifunctional activities mediated by the ENDO180 receptor (e.g. internalization activity, Ca²⁺ dependent lectin activity, collagen binding, plasminogen activation). In a further embodiment, measurement of step (i) is carried out in cells expressing the ENDO1 80 receptor.

In yet another embodiment of the invention, the parameter measured in step (i) of said processes is fibroblast adhesion in the absence and in the presence of the compound, and determining either in step (ii) or in step (iii), according to the process used, whether the adhesion of fibroblast is affected by said compound.

The cells contacted with the compound may be renal fibroblasts and the activity measured in step (i) may be collagen deposition in the extracellular matrix of said renal fibroblasts. The cells contacted with the compound may also be renal tubular cells and the activity that would be measured in step (i) is proliferation of said renal tubular cells.

The cells being contacted with the compound may be cells that express the ENDO180 receptor naturally, or cells that have previously been transfected by the ENDO180 gene, either transiently or stably transfected. In a further embodiment, the cells used in the above mentioned processes have a mutated inactive form of the ENDO180 gene as a control.

The present invention further provides for a process for identifying a compound capable of modulating nephropathy, CRI or CRF and preferably diabetic nephropathy, via a human ENDO180 receptor, and/or of modulating osteoporosis or osteoarthritis, wherein the compound modulates the activity of the ENDO180 receptor at least 2-fold, more preferably at least 10-fold, and even more preferably at least 100-fold, more effectively than it modulates the activity of one or more of the set of urokinase related proteins or receptors and mannose receptors consisting of uPA, uPAR, PLA₂R, DEC-205M6-gp200 and mannose receptor.

The present invention further provides transgenic animals carrying at least one expressible gene that encodes the ENDO180 receptor or domains thereof, identified by the present invention under kidney specific promoter.

The present invention provides a process for discovering drugs for use in treating nephropathy, CRI or CRF, in a patient in need of such treatment. These drugs, in therapeutically effective amounts, will affect, preferably enhance, the activity of the ENDO180 receptor, as encoded by the nucleic acid sequences or as presented by the amino acid sequences identified herein, or by the probes of the present invention.

It is in the scope of the present invention, to use a compound identified according to the processes described herein, in the preparation of a medicament for therapy of fibrosis, nephropathy, CRI or CRF and more specifically for therapy of diabetic nephropathy, kidney fibrosis and glomerulosclerosis, or for therapy of osteoporosis or osteoarthritis in a patient in need of such treatment.

It should be noticed that although these drugs are preferentially directed to treatment of kidney fibrosis, they may also be useful for the treatment of other fibrotic diseases, such as liver, lung and heart. These drugs may also be used to treat or prevent restenosis, i.e., to prevent or reduce proliferation of smooth muscle cells. These drugs may also be used as anti-angiogenic drugs for the treatment of cancer and other conditions where preventing or reducing proliferation of endothelial cells is desired. These drugs may also be used for treatment of osteoarthritis and treatment of osteoporosis as well as other bone diseases.

Any of the screening assays according to the present invention can include a step of identifying the compound (as described above) which tests positive in the assay, and can also include the further step of producing said compound as a medicament. It can also include steps of improving the compound to increase its desired activity before incorporating the improved compound into a medicament. It is considered that medicaments comprising such compounds are part of the present invention. This invention further provides for the use of a compound which modulates the ENDO 180 gene or polypeptide in the preparation of a medicament for treatment of disease in a patient in need of such treatment. The disease may be fibrosis, nephropathy, CRI or CRF and more specifically diabetic nephropathy, kidney fibrosis and glomerulosclerosis; the disease may also be osteoporosis or osteoarthritis.

The present invention further provides for a process of preparing a pharmaceutical composition which comprises:
(i) identifying a compound that modulates activity of a human ENDO180 receptor using any of the processes of the above; and
(ii) admixing said compound with a pharmaceutically acceptable carrier, wherein the compound admixed with the carrier is present in a pharmaceutically effective amount.

Additionally, the present invention provides a method of regulating a pathology or disease in a patient in need of such treatment by administering to a patient a therapeutically effective amount of at least one antisense (AS) oligonucleotide or at least one siRNA against the nucleic acid sequences or a dominant negative peptide directed against the ENDO180 sequences or ENDO180 proteins or an antibody directed against the ENDO180 polypeptide. Preferably the pathology is a fibrosis-associated pathology, eg nephropathy, CRI or CRF and more specifically diabetic nephropathy, kidney fibrosis and glomerulosclerosis, but it may also be osteoporosis or osteoarthritis.

As used herein, “negative dominant peptide” refers to a partial cDNA sequence that encodes a part of a protein, i.e., a peptide (Herskowitz I. (1987) Nature (Review) 329(6136): 219-222). This peptide can have a function different from that of the protein from which it was derived. It can interact with a wild type protein target and inhibit its activity or it can interact with other proteins and inhibit their activity in response to the wild type target protein. Specifically, negative dominant refers to the ability of a peptide to inhibit the activity of a natural protein normally found in the cell in order to modulate the cellular phenotype, i.e., making the cell more resistant or sensitive to killing. For therapeutic intervention either the peptide itself is delivered as the active ingredient of a pharmaceutical composition or the cDNA can be delivered to the cell utilizing the same methods as for AS delivery.

The modulator is dosed and delivered in a pharmaceutically acceptable carrier as described herein below. The modulator may be an antagonist agent or regulating active ingredient As used herein, the term “antagonist or antagonizing” is understood in its broadest sense. Antagonism can include any mechanism or treatment that results in inhibition, inactivation, blocking or reduction in gene activity or gene product. It should be noted that the inhibition of a gene or gene product may provide for an increase in a corresponding function that the gene or gene product was regulating. The antagonizing step can include blocking cellular receptors for the gene products and can include AS or siRNA treatment as discussed below.

The compounds (modulators) or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art. The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention and they include liposomes and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24-to 48 hrs or by continuous infusion during a period of 1-2 weeks or longer.

Many reviews have covered the main aspects of AS technology and its enormous therapeutic potential (Anazodo et al. (1995) Gene 166(2):227-232). There are reviews on the chemical aspects (Crooke S T (1995)Hematol Pathol. (Review) 9(2):59-72; Uhimarn et al.(2000) Methods Enzymol. 313:268-284.), cellular aspects (Wagner R W (1994) Nature (Review) 372(6504):333-335), and therapeutic aspects Hanania et al. (1995) Am J Med.(Review) 99(5):537-552; Scanlon et al. (1995) FASEB J. (Review) 9(13):1288-1296; Gewirtz AM (1993) Leuk Lymphoma 1993;11 Suppl 1:131-137) of this rapidly developing technology.

RNA interference (siRNA or RNAi) technology may also be used in the methods of this invention. By “silencing RNA” (siRNA) is meant an RNA molecule which decreases or silences (prevents) the expression of a gene/mRNA of its endogenous or cellular counterpart. The term is understood to encompass “RNA interference” (RNAi), and “double-stranded RNA” (dsRNA). For recent information on these terms and proposed mechanisms, see Bernstein E., Denli A M., Hannon G J: The rest is silence. RNA. November 2001;7(11):1509-21; Nishikura K.: A short primer on RNAi: RNA-directed RNA polymerase acts as a key catalyst. Cell. Nov. 16, 2001;107(4):415-8; and PCT publication WO 01/36646 (Glover et al).

Delivery systems aimed specifically at the enhanced and improved delivery of siRNA into mammalian cells have been developed, see, for example, Shen et al (FIBS letters 539: 111-114 (2003)), Xia et al., Nature Biotechnology 20: 1006-1010 (2002), Reich et al., Molecular Vision 9: 210-216 (2003), Sorensen et al. (J. Mol. Biol. 327: 761-766 (2003), Lewis et al., Nature Genetics 32: 107-108 (2002) and Simeoni et al., Nuclaic Acids Research 31, 11: 2717-2724 (2003). siRNA has recently been successfully used for inhibition in primates; for further details see Tolentino et al., Retina 24(1) February 2004 pp 132-138.

Modifications or analogs of nucleotides can be introduced to improve the therapeutic properties of the nucleotides. Improved properties include increased nuclease resistance and/or increased ability to permeate cell membranes.

Accordingly, the present invention also includes all analogs of, or modifications to, a polynucleotide or oligonucleotide of the invention that does not substantially affect the function of the polynucleotide or oligonucleotide. The nucleotides can be selected from naturally occurring or synthetically modified bases. Naturally occurring bases include adenine, guanine, cytosine, thymine and uracil. Modified bases of the oligonucleotides include xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogs of nucleotides can be prepared wherein the structures of the nucleotides are fundamentally altered and are better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone similar to that found in peptides. PNA analogs have been shown to be resistant to degradation by enzymes and to have extended lives in vivo and in vitro, Further, PNAs have been shown to bind more strongly to a complementary DNA sequence than to a DNA molecule. This observation is attributed to the lack of charge repulsion between the PNA strand and the DNA strand. Other modifications that can be made to oligonucleotides include polymer backbones, cyclic backbones, or acyclic backbones.

The active ingredients of the pharmaceutical composition can include oligonucleotides that are nuclease resistant, needed for the practice of the invention, or a fragment thereof shown to have the same effect targeted against the appropriate sequence(s) and/or ribozymes. Combinations of active ingredients as disclosed in the present invention can be used, including combinations of AS sequences.

The AS oligonucleotides, ribozymes, siRNA and cDNA of the present invention can be synthesized by any method known in the art for ribonucleic or deoxyribonucleic nucleotides. For example, a commercial polynucleotide synthesizer (eg Applied Biosystems 380B DNA synthesizer) can be used. When fragments are used, two or more such sequences can be synthesized and linked together for use in the present invention.

The nucleotide sequences of the present invention can be delivered either directly or via viral or non-viral vectors. When delivered directly the sequences are generally rendered nuclease resistant. Alternatively the sequences can be incorporated into expression cassettes or constructs such that the sequence is expressed in the cell as discussed herein below. Generally the construct contains the proper regulatory sequence or promoter to allow the sequence to be expressed in the targeted cell.

In one embodiment of the invention, the promoter linked to the coding region of the ENDO180 is the KSP promoter, known to be specifically expressed in the kidneys.

The polypeptides of the present invention may be produced recombinantly (see generally Marshak et al., 1996 “Strategies for Protein Purification and Characterization. A laboratory course manual.” Plainview, N.Y.: Cold Spring Harbor Laboratory Press, 1996) and analogs may be produced by post-translational processing. Differences in glycosylation can provide polypeptide analogs.

As used herein, the term “polypeptide” refers to, in addition to a polypeptide, a peptide and a fill protein. As used herein, “biological function” refers to the biological property of the molecule and in this context means an in vivo effector or antigenic function or activity that is directly or indirectly performed by a naturally occurring polypeptide or nucleic acid molecule. Biological functions include but are not limited to receptor binding, any enzymatic activity or enzyme modulatory activity, any carrier binding activity, any hormonal activity, any activity in internalizing molecules or translocation from one compartment to another, any activity in promoting or inhibiting adhesion of cells to extracellular matrix or cell surface molecules, or any structural role, as well as having the nucleic acid sequence encode functional protein and be expressible. The antigenic functions essentially mean the possession of an epitope or an antigenic site that is capable of cross-reacting with antibodies raised against a naturally occurring protein. Biologically active analogs share an effector function of the native polypeptide that may, but need not, in addition possess an antigenic function.

This application is also directed to a method of diagnosing a disease such as fibrosis, nephropathy, CRF, CRI and, preferably diabetic nephropathy or kidney fibrosis, or osteoporosis or osteoarthritis, in a subject, comprising determining in a sample from the subject the level of an ENDO180 receptor polypeptide, wherein a higher level of the polypeptide compared to the level in a subject free of the disease is indicative of the disease. In preferred embodiments the ENDO180 receptor comprises consecutive amino acids, the sequence of which is set forth in SEQ ID NO:2.

In diagnosis, the sample may be taken from a bodily fluid or from a tissue, preferably kidney tissue; the bodily fluid is selected from the group of fluid consisting of blood, lymph fluid, ascites, serous fluid, pleural effusion, sputum, cerebrospinal fluid, lacrimal fluid, synovial fluid, saliva, stool, sperm and urine, preferably blood or urine.

Measurement of level of the ENDO180 polypeptide may be determined by a method selected from the group consisting of immunohistochemistry, western blotting, ELISA, antibody microarray hybridization and targeted molecular imaging. Such methods are well-known in the art, for example for immunohistochemistry: M. A. Hayat (2002) Microscopy, Immunohistochemistry and Antigen Retrieval Methods: For Light and Electron Microscopy, Kluwer Academic Publishers; Brown C (1998): “Antigen retrieval methods for immunohistochemistry”, Toxicol Pathol; 26(6): 830-1); for western blotting: Laemmeli U K (1970): “Cleavage of structural proteins during the assembley of the head of a bacteriophage T4”, Nature;227: 680-685; and Egger & Bienz(1994) “Protein (western) blotting”, Mol Biotechnol; 1(3): 289-305); for ELISA: Onorato et al.(1998) “Immunohistochemical and ELISA assays for biomarkers of oxidative stress in aging and disease”, Ann NY Acad Sci 20; 854: 277-90); for antibody microarray hybridization:Huang(2001) “Detection of multiple proteins in an antibody-based protein microarray system, Immunol Methods 1; 255 (1-2): 1-13); and for targeted molecular imaging: Thomas (2001). Targeted Molecular Imaging in Oncology, Kim et al (Eds)., Springer Verlag inter alia.

Measurement of level of ENDO180 polynucleotide may be determined by a method selected from: RT-PCR analysis, in-situ hybridization, polynucleotide microarray and Northern blotting. Such methods are well-known in the art, for example for in-situ hybridization Andreeff & Pinkel (Editors) (1999), “Introduction to Fluorescence In Situ Hybridization: Principles and Clinical Applications”, John Wiley & Sons Inc.; and for Northern blotting Trayhurn (1996) “Northern blotting”, Proc Nutr Soc; 55(1B): 583-9 and Shifman & Stein (1995) “A reliable and sensitive method for non-radioactive Northern blot analysis of nerve growth factor mRNA from brain tissues”, Journal of Neuroscience Methods; 59: 205-208 inter alia.

The present invention is also directed to a process of identifying a compound capable of modulating the activity of a human ENDO180 receptor by screening a plurality of compounds that comprises the steps of:
(i) measuring the binding of the ENDO180 receptor to an interactor with which the ENDO180 receptor interacts specifically in vivo in the absence or presence of a plurality of compound; and
(ii) determining whether the binding of the ENDO180 receptor to said interactor is affected by the presence of the plurality of compounds; and
(iii) separately determining which compound or compounds present in the plurality modulates the binding of the human ENDO180 receptor.

The above discussion provides a factual basis for the use of the sequences of the present invention to identify CRF, CRI and nephropathy-regulated genes and provide diagnostic probes. The methods employed and the utility of the present invention are demonstrated by the following non-limiting examples.

### METHODS

General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and MnRNA sequences (Testoni et al., 1996, Blood 87:3822.)

General Methods in Immunology

Standard methods in immunology known in the art and not specifically described are generally followed as in Stites et al (eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Immunoassays

In general ELISAs, where appropriate, are one type of immunoassay employed to assess a specimen. ELISA assays are well known to those skilled in the art. Both polyclonal and monoclonal antibodies can be used in the assays. Where appropriate other immunoassays, such as radioimmunoassays (RIA) can be used as are known to those skilled in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al., Molecular Cloning: A Laboratory Manual, Cold Springs Harbor, New York, 1989.

Antibody Production

By the term “antibody” as used in the present invention is meant both poly- and mono-clonal complete antibodies as well as fragments thereof, such as Fab, F(ab′)2, and Fv, which are capable of binding the epitopic determinant. These antibody fragments retain the ability to selectively bind with its antigen or receptor and are exemplified as follows, inter alia:
(1) Fab, the fragment which contains a monovalent antigen-binding fragment of an antibody molecule can be produced by digestion of whole antibody with the enzyme papain to yield a light chain and a portion of the heavy chain;
(2) (Fab′)2, the fragment of the antibody that can be obtained by treating whole antibody with the enzyme pepsin without subsequent reduction; F(ab′2) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

Such fragments having antibody functional activity can be prepared by methods known to those skilled in the art (Bird et al. (1988) Science 242:423-426)

Conveniently, antibodies may be prepared against the immunogen or portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art, as described generally in Harlow and Lane (1988), Antibodies: A Laboratory Manual, Cold Spring Harbor Laboratory, Cold Spring Harbor, N.Y., and Borrebaeck (1992), Antibody Engineering—A Practical Guide, W.H. Freeman and Co., NY.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific; that is, the sera can be absorbed against related immunogens so that no cross-reactive antibodies remain in the sera, rendering it monospecific.

For producing monoclonal antibodies the technique involves hyperimmunization of an appropriate donor with the immunogen, generally a mouse, and isolation of splenic antibody-producing cells. These cells are fused to an immortal cell, such as a myeloma cell, to provide a fused cell hybrid that is immortal and secretes the required antibody. The cells are then cultured, in bulk, and the monoclonal antibodies harvested from the culture media for use.

For producing recombinant antibody see generally Huston et al. (1991) “Protein engineering of single-chain Fv analogs and fusion proteins” in Methods in Enzymology (J J Langone, ed., Academic Press, New York, N.Y.) 203:46-88; Johnson and Bird (1991) “Construction of single-chain Fvb derivatives of monoclonal antibodies and their production in Escherichia coli in Methods in Enzymology (J J Langone, ed.; Academic Press, New York, N.Y.) 203:88-99; Mernaugh and Memaugh (1995) “An overview of phage-displayed recombinant antibodies” in Molecular Methods In Plant Pathology (R P Singh and U S Singh, eds.; CRC Press Inc., Boca Raton, Fla.:359-365). Additionally, messenger RNAs from antibody-producing B-lymphocytes of animals, or hybridoma can be reverse-transcribed to obtain complementary DNAs (cDNAs). Antibody cDNA, which can be full or partial length, is amplified and cloned into a phage or a plasmnid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe (1982.), Immunochemistry in Practice, Blackwell Scientific Publications, Oxford). The binding of antibodies to a solid support substrate is also well known in the art (for a general discussion, see Harlow & Lane (1988) Antibodies: A Laboratory Manual, Cold Spring Harbor Laboratory Publications, New York; and Borrebaeck (1992), Antibody Engineering—A Practical Guide, W.H. Freeman and Co.). The detectable moieties contemplated with the present invention can include, but are not limited to, fluorescent, metallic, enzymatic and radioactive markers such as biotin, gold, ferritin, alkaline phosphatase, P-galactosidase, peroxidase, urease, fluorescein, rhodamine, tritium, ¹⁴C and iodination.

Recombinant Protein Purification

For standard purification, see Marshak et al. (1996), “Strategies for Protein Purification and Characterization. A laboratory course manual.” CSHL Press.

Transgenic and Knockout Methods

The present invention provides for a transgenic gene and a polymorphic gene animal and cellular (cell line) model, as well as for a knockout model. These models are constructed using standard methods known in the art and as set forth in U.S. Pat. Nos. 5,487,992; 5,464,764; 5,387,742; 5,360,735; 5,347,075; 5,298,422; 5,288,846; 5,221,778; 5,175,385; 5,175,384; 5,175,383; 4,736,866; as well as Burke and Olson (1991) “Preparation of Clone Libraries in Yeast Artificial-Chromosome Vectors” in Methods in Enzymology, 194, “Guide to Yeast Genetics and Molecular Biology”, eds. C. Guthrie and G. Fink, Academic Press, Inc., Chap. 17:251-270; Capecchi (1989) “Altering the genome by homologous recombination”, Science, 244:1288-1292; Davies et al. (1992) “Targeted alterations in yeast artificial chromosomes for inter-species gene transfer”, Nucleic Acids Research, 20 (11): 2693-2698; Dickinson et al. (1993) “High frequency gene targeting using insertional vectors”, Human Molecular Genetics, 2(8):1299-1302; Duff and Lincoln (1995) “Insertion of a pathogenic mutation into a yeast artificial chromosome containing the human APP gene and expression in ES cells”, Research Advances in Alzheimer's Disease and Related Disorders Khalid Iqbal (Editor), James A. Mortimer (Editor), Bengt Winblad (Editor), Henry M. Wisniewski (Editor); Huxley et al. (1991) “The human HPRT gene on a yeast artificial chromosome is functional when transferred to mouse cells by cell fusion”, Genomics, 9:742-750; Jakobovits et al. (1993) “Germ-line transmission and expression of a human-derived yeast artificial chromosome”, Nature, 362: 255-261; Lamb et al. (1993) “Introduction and expression of the 400 kilobase precursor amyloid protein gene in transgenic mice”, Nature Genetics, 5:22-29; Pearson and Choi (1993) Expression of the human b-amyloid precursor protein gene from a yeast artificial chromosome in transgenic mice. Proc. Natl. Acad. Sci. (USA), 90:10578-10582; Rothstein, (1991) “Targeting, disruption, replacement, and allele rescue: integrative DNA transformation in yeast” in Methods in Enzymology, 194, “Guide to Yeast Genetics and Molecular Biology”, eds. C. Guthrie and G. Fink, Academic Press, Inc., NY, Chap. 19:281-301; Schedl et al. (1993) “A yeast artificial chromosome covering the tyrosinase gene confers copy number-dependent expression in transgenic mice”, Nature, 362:258-261; Strauss et al. (1993) “Germ line transmission of a yeast artificial chromosome spanning the murine a₁ (1) collagen locus”, Science, 259:1904-1907. Additionally, PCT patent applications WO 94/23049, WO 93/14200, WO 94/06908, WO 94/28123 also provide information.

### EXAMPLES

### Example 1

### Identification of ENDO180 Overexpression by Microarray Hybridization Study

In accordance with the present invention, the microarray hybridization approach was utilized in order to discover genes that are differentially regulated in diabetic nephropathy and kidney fibrosis.

Microarray-based analysis of gene expression was based on the analysis of human fibroblasts subject to selected stimuli resulting in changes in extracellular collagen accumulation and proliferation—the hallmarks of fibrosis. According to the present invention, a specific “Fibrosis” DNA chip was first prepared followed by a microarray hybridization experiments with 19 different types of probes. Analysis of the results was carried out by proprietary algorithms, and analysis of the selected set of genes was performed by the inventors using bioinformatics and the scientific literature.

Preparation of Specific “Fibrosis” DNA Chip

A dedicated human “Fibrosis” DNA chip was prepared according to assignee's SDGI method (PCT Application Publication No. WO 01/75180), from growth-arrested human fibroblasts. Growth arrest was imposed by the treatments presented in Table 1 below:

TABLE 1
Biological material for “Fibrosis” chip preparation

| | Treatment |
|---|---|
| 1 | G1 arrested serum-starved l.p. HFs* |
| 2 | l.p. HFs* 36 hr and 48 hr following 8Gy γ-irradiation |
| 3 | l.p. HFs* 5 days after addition of H₂0₂ 200 μM |
| 4 | l.p. HFs* following UV (growth-arresting dose) |
| 5 | l.p. HFs* 48 hr following Bleomycin treatment 50 ng/ml |
| 6 | l.p. HFs* 48 hr following Etoposide treatment 400 ng/ml |
| 7 | l.p. HFs* 48 hr following Adriamycin treatment 50 ng/ml |
| 8 | Senescent HFs from normal individuals |
| 9 | Senescent HFs from individuals with Werner syndrome |
| 10 | Senescent HFs from individuals with Progeria |

l.p. HF* - low passage human fibroblasts

Unless indicated otherwise, all human fibroblasts (HFs) were at passage 15 prior to treatment. RNA from all treated HFs was prepared, pooled and used for library preparation by the proprietary SDGI method of the assignee. This chip also contained human ESTs coding for genes known to play a part in apoptosis, cytotoxicity and replicative cellular senescence.

Fibroblast Cultivation

Normal human fetal lung fibroblasts (WI-38, Coriell Cell Repositories) were cultured and sub-cultured in DMEM, supplemented with 10% inactivated fetal bovine serum (FBS), 2 mM L-glutamine, 100 U/ml penicillin, 100 μg/ml streptomycin. Fibroblasts were grown to confluence in 25 cm² tissue flasks and sub-cultured after trypsinization (0.5% trypsin-EDTA in Hank's balanced solution without Ca²⁺ and Mg²⁺) at 37° C. in an atmosphere of 5% CO₂. Two ml of trypsin were added to each flask and incubated for 5 min; then cultures were centrifuged (5 min, 1000 rpm) and fresh medium was added to the pellet. Splitting conditions were 1:4-1:6.

Since the hallmarks of fibrotic disease are fibroblast proliferation and/or enhanced synthesis of extracellular matrix components (mainly collagen), different treatment regimes were used and the rates of both proliferation and collagen synthesis by the treated fibroblasts cultured in vitro was examined.

Fibroblast Proliferation Assay

The proliferation rate of sub-confluent fibroblasts was evaluated by staining with neutral red (BioRad). Fibroblasts were seeded in 96-well plate (6×10³/well) in 200 μl of supplemented DMEM/10% FBS. After overnight culture, wells were washed twice with supplemented DMEM/2% FBS. Then, either TGF-P (2-20 ng/ml) or deferoxamine mesylate (DFO, which leads to conditions of chemical hypoxia) at a concentration of 100 mM was added in 200 μl of supplemented DMEM/2% FBS for either 16 hours, 24 hours, 72 hours, or 5 days.

In the case of glucose treatments, after overnight culture, cell-containing wells were washed twice with supplemented glucose-free DMEW2% PBS. Working concentrations of glucose (5.5 mM, 15 mM, 27.5 mM, or 55 mM) were prepared by dissolving stock solution (110 mM) in supplemented DMEM without glucose/2% FBS. Prepared solutions of glucose were added to fibroblast cultures for either 24 or 72 hours.

Upon completion of incubation, cells were stained with 100 μl of 1% neutral red for 2 hours. After washing with cold PBS, fibroblast monolayers were fixed with 200 μl of ethanol-Sorenson buffer solution (1:1) for 10 minutes. Optical density was measured with an automated spectrophotometer (λ=540 nm).

Collagen Production Assay

Collagen production by confluent fibroblast monolayers was assessed by [³H]-proline incorporation into collagenous proteins. Fibroblasts were seeded in 24-well tissue culture plates (2×10⁴/well) and grown in 1 ml of supplemented DMEA10% FBS until confluence.

Confluent fibroblast cultures were incubated with prepared solutions for either 24 or 48 hours. Then [³H]-proline (10 μCi/well) was added and cultures were incubated for an additional 24 hours. At the end of the incubation, medium was decanted and incubated with or without collagenase for 18 hours, followed by precipitation with 50% and 10% TCA. The production of collagen was determined as the difference between total [³H]proline-containing proteins in the sample incubated without collagenase and those left after collagenase digestion. To determine the number of cells in each well, fibroblasts were detached by trypsinization on the last day of the experiment, and counted in a hemocytometer.

Probes for microarray hybridization were derived from these treated fibroblasts. In accordance with the present invention, treatments that are relevant for diabetic nephropathy development were used, such as glucose deprivation or hypoxia (modeling ischemic conditions that develop in fibrotic kidney), high glucose (modeling diabetic hyperglycemia) and TGF-β induction (modeling a fibrotic condition that is characterized by growth factor and cytoline imbalance).

More specifically, human fibroblasts were treated as followed:
1. glucose at 4 different concentrations (5.5, 15, 27.5, or 55 mM) for 24 and 72 hours
2. TGF-β at 2-20 ng/ml, for 24 or 72 hours
3. DFO deferoxamine at a concentration of 100 mM, dissolved in 0.5 ml of DMEM, containing 5% FCS, 50 μg/ml β-aminoproprionitrile, and 50 βg/ml ascorbic acid (modified DMEM), for 24, 48 and 72 hours.

The analysis of proliferation rate of these cultured fibroblasts showed that cultivation of fibroblasts for 24 hrs in glucose-free medium and in 55 mM glucose resulted in a decrease of their proliferation rate by 20% and 30%, respectively, compared to control cultures. Addition of glucose at different concentrations (from 5.5 mM to 27.5 mM) practically did not affect fibroblast proliferation compared to the control. A significant decrease in fibroblast proliferation was observed after addition of DFO (from 20% decrease after 16 hours incubation to 80% decrease after 5 days of treatment). TGF-β, added at concentrations of 2 and 20 ng/ml, led to an increase in the fibroblast proliferation rate by ˜60% after 24 hours treatment.

As for collagen synthesis rate, all treatments (except for 55 mM glucose) led to increased collagen production by fibroblasts. The most significant effect was observed after addition of TGF-β at concentrations of 2-20 ng/ml, providing enhancement of collagen production by 110-180%.

In the next step, the RNA from these treated fibroblasts was extracted and used for preparation of probes for microarray hybridization. The scheme of hybridization is presented below:

TABLE 2
Hybridization scheme

| Probe | | | Probe | | |
|---|---|---|---|---|---|
| name | Dye | PROBE 1 | name | Dye | Probe 2 |
| FG1A | Cy3 | Untreated | FG1B | Cy5 | l.p. untreated HFs* |
| FG19A | | human | FG19B | | l.p. untreated HFs* |
| FG18A | | fibroblasts- | FG18B | | l.p. HFs* w/o |
| | | Common | | | glucose 72 hr |
| FG17A | | Normalizing | FG17B | | l.p. HFs* TGF-β |
| | | Probe | | | 20 ng/μl 72 h |
| FG16A | | | FG16B | | l.p. HFs* TGF-β |
| | | | | | 20 ng/μl 24 h |
| FG15A | | | FG15B | | l.p. HFs* w/o glucose |
| | | | | | 24 h |
| FG14A | | | FG14B | | l.p. HFs* TGF-β |
| | | | | | 2 ng/μl 72 h |
| FG13A | | | FG13B | | l.p. HFs* TGF-β |
| | | | | | 2 ng/ml 24 h |
| FG12A | | | FG12B | | l.p. HFs* 5.5 mM |
| | | | | | glucose 72 h |
| FG11A | | | FG11B | | l.p. HFs* 5.5 mM |
| | | | | | glucose 24 h |
| FG10A | | | FG10B | | l.p. HFs* Hypoxia 5 days |
| FG9A | | | FG9B | | l.p. HFs* 55 mM |
| | | | | | glucose 72 h |
| FG8A | | | FG8B | | l.p. HFs* 55 mM |
| | | | | | Glucose 24 h |
| FG7A | | | FG7B | | l.p. HFs* Hypoxia 3 days |
| FG6A | | | FG6B | | l.p. HFs* 27.5 mM |
| | | | | | Glucose 72 h |
| FG5A | | | FG5B | | l.p. HFs* 27.5 mM |
| | | | | | glucose 24 h |
| FG4A | | | FG4B | | l.p. HFs* hypoxia 16 h |
| FG3A | | | FG3B | | l.p. HFs* 15 mM |
| | | | | | glucose 72 h |
| FG2A | | | FG2B | | l.p. HFs* 15 mM |
| | | | | | glucose 24 h |

l.p. HFs* = low passage human fibroblasts

Probe 1 was identical in all hybridization experiments, and was produced with RNA extracted from untreated human fibroblasts (passage 15). This probe served both as a biological control and as a common normalizing probe that allowed comparison of results obtained from different hybridization experiments.

In accordance with the present invention, a total of 19 hybridization experiments were performed. In two hybridization experiments (FG1 and FG19), the common normalizing probe (Probe 1 in all hybridization experiments) was hybridized against itself (i.e., Probe 1 was identical to Probe 2). In general, these hybridization experiments were conducted in order to determine labeling quality and to evaluate the ability of the common normalizing probe to detect most of the cDNA clones printed on the chip.

Bioinformatics Analysis of Gene Expression Results

The proprietary statistical analysis of the assignee of microarray hybridization results was based on the assumption that changes in gene expression correlate with different physiological and pathological conditions and, in many instances, underlie them. Thus, in a given set of experiments, a certain treatment regime/condition is associated with a particular gene expression profile. Furthermore, the inventors assumed that some hierarchy exists among the different pathological conditions/physiological treatments, i.e., some are more similar than others.

The final goal of such an analysis was to elucidate both specific and general mechanisms underlying complex biological phenomena by comparison of gene expression patterns within a large panel of conditions, each representing some of its aspects. More specifically, in the set of hybridization results generated in accordance with the present invention, the inventors anticipated observing groups of genes the expression of which was either common or unique to different types of conditions relevant to diabetic nephropathy (hypoxia, high glucose, TGF-β), and wherein the response to the applied treatment was either acute or chronic.

Results of Hybridization Analysis

In accordance with the present invention, in human fibroblasts differentially treated in vitro, a set of 46 genes was identified, the activity of which was significantly up-regulated by various types of applied treatments.

The identified gene products fell into nine distinct functional groups:
1. Extracellular matrix proteins and receptors to extracellular matrix proteins;
2. Secreted growth factor interacting proteins and potential growth factor receptors;
3. Signal transduction adaptor proteins;
4. Cytoskeletal proteins (mostly related to actin cytoskeleton function);
5. Ca²⁺-binding proteins;
6. ER-resident proteins;
7. Nuclear import mediators;
8. Proteins involved in RNA and protein synthesis and processing;
9. Novel genes;

The 46 up-regulated genes identified were divided as follows:
(a) 11 were known genes with known functions with recognized involvement in fibrosis (collagens type III and I (α1 and α2), fibronectin, decorin, β-ig-h3, integrin, TIMP3, CD44, smooth muscle actin, and Arp2/3 (Arc34);
(b) 28 were known genes with known function but with previously unknown involvement in fibrosis. ENDO180, the subject of the present application, falls into this category;
(c) 2 were genes coding for proteins with unknown function and unknown involvement in fibrosis, and;
(d) 5 were novel genes.

Using the microarray hybridization technique it was found that the expression of ENDO180 has been induced by TGF-β treatment of human fibroblasts by at least 3-fold.

### Example 2

### Construction of Kidney Specific Promoter for Transzenic Mice

The KSP-cadherin gene promoter (3593bp) which is known to be tubular specific (epithelial cells specific) has been cloned in the pMCSZ vector which contains lacZ.

Trangenic expression of lacZ reporter gene controlled by the kidney-specific cadherin promoter was evaluated in transient transgenic mouse embryos. 1 out of 9 El 8.5 embryos and 2 out of 8 E15.5 embryos showed specific expression pattern in the kidneys (the expression in the E15.5 kidneys was much weaker). The expression was located towards the medullary region, in the center of the metanephros (a wholemount staining).

Analysis of sections of wholemount stained kidneys of the E18.5 embryo revealed the transgene expression in tubular epithelial cells, which according to their location and characteristic branching seem to be the collecting ducts. Expression was also evident throughout the urether. The collecting ducts develop from the branching ureteric bud, and at this stage of development, the majority of the collecting duct system is contained in the medullary region of the metanephros. No expression was observed in the subcapsular nephrogenic zone.

These results are in correlation with those described by Igarashi et al. 1999 (Am. J. Physiol. 277 (4 pt 2). By co-labeling the lacZ expressing cells with Dolichos biflorns agglutinin, a lectin that specifically labels the collecting ducts and uretheric buds, they identified the lacZ expressing cells as epithelial tubular cells. They also reported that the expression of the transgene increases during gestation (similar to our findings) and remains high in the adult kidney (yet to be confirmed).

In conclusion, we found that the KSP-Cadherin promoter is a specific promoter for kidney epithelial cell expression.

### Example 3

### Construction of Transgenic Mice Expressing Fibronectin (Fa) Domain of ENDO180 for in vivo Validation of ENDO180 Activity in Mouse Kidneys

A DNA fragment of 682 base pairs from position 105-787 of the mouse mannose receptor, ENDO180 (gi. 6678933), was excised from mouse DNA; this fragment is the fibronectin (FN) domain The beta-actin kozak sequence was added to the 5′ end and the flag tag sequence to the 3′ end.

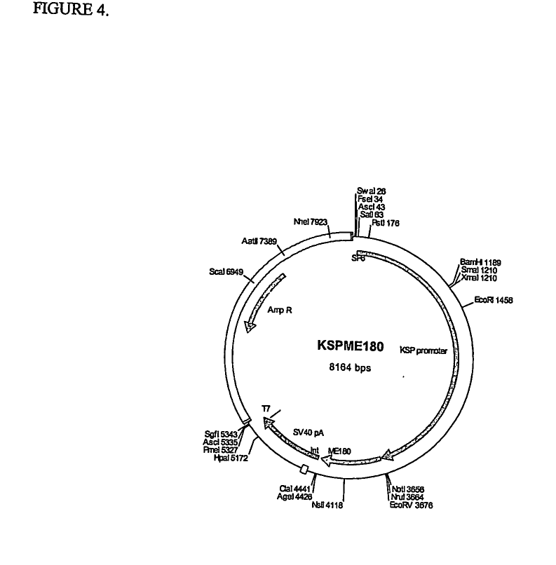

The gene was cloned into a plasmid containing the KSP promoter, and the 5.3 Kb Asc1 fragment was excised, purified and injected into mouse eggs (See FIG. 4).

Out of 42 pups born, 9 were found to carry the ENDO180 gene. RNA from these lines was analysed for expression in the kidney both by RNA and protein analysis.

Expression of transgene was verified in line number 4 which was chosen for expansion. The derivative mice are used for evaluation of fibrosis development following UUO experiment or 5 by aging.

### Example 4

### Assessment of in vivo Models for Kidney Fibrosis by Morphology, Immunostaining and in situ Hybridization

Morphology

To assess general morphology, paraffin kidney sections were stained by hematoxilin-eosin (HE). Sirius Red (SR) staining was used to reveal collagen in the sections.

Immunostaining

Accumulation of interstitial myofibroblasts is regarded as an important initial step in the development of the renal fibrotic process. To reveal myofibroblasts, monoclonal antibody specific to α-smooth muscle actin (clone 1A4) was used for the peroxidase-antiperoxidase (PAP) immunostaining of kidney paraffin sections. The monoclonal antibody PC-10 was used for the immmunostaining of proliferating cell nuclear antigen (PCNA). To achieve adequate PCNA immunostaining, de-paraffinized sections were subjected to antigen retrieval procedure before performing PAP staining.

In situ Hybridization

³⁵S-labeled riboprobes were synthesized and hybridized to kidney paraffin sections according to standard protocol. After the post-hybridization washing step, sections were air-dried and macro-autoradiography was performed by exposing the slides to X-ray film overnight. For micro-autoradiography, slides were dipped into nuclear track emulsion and stored in darkness at 4° C. Exposed slides were developed after 2-3 weeks and sections were slightly counter-stained with HE and cover-slipped for microscopic examination.

Probes for in situ Hybridization

The cDNAs used as the templates for riboprobe synthesis were rat osteopontin cDNA, mouse transforming growth factor β1 cDNA, mouse procollagen α1(I) cDNA and mouse thrombospondin1 cDNA.

Examples of Models

ZDF Rats

Samples of 9-month-old ZDF rats (Zucker diabetic fatty rats) presented hydronephrotic kidneys with dilated calyces. Microscopically these samples presented a picture of glomerulosclerosis and tubulointerstitial fibrosis. In accordance with these morphological changes, the expression of marker genes as measured by in situ hybridization (osteopontin (OPN), transforming growth factor β1 (TGF-β1) and procollagen α1(I) (Col1)) was significantly changed when compared to normal kidneys. Strong OPN expression was detectable in all tubular structures in both cortex and medulla. The TGF-β1 expression was widespread throughout interstitial cells. Some epithelial cells also showed TGF-β1 expression. Col1 expression was detectable by in situ hybridization in most interstitial cells within the medulla, while cortical expression was “focal”.

Aged fa/fa (Obese Zuckers Rats

Samples of 12-month-old fa/fa rats presented strong glomerulosclerosis and diffuse tubulointerstitial fibrosis throughout the cortex and the medulla. The pattern of marker gene expression corresponded to morphological changes. OPN was expressed by tubular structures in the cortex and the medulla. Multiple interstitial cells expressed TGF-β1. Significantly, multiple foci and single interstitial cells showed strong Col1 expression in both cortex and medulla so that the number of Col1-expressing cells appeared to be higher in fa/fa samples than in ZDF samples.

Interestingly, Col1 expression was not detected in glomeruli of either ZDF or fa/fa rats in spite of the prominent accumulation of collagen, as revealed by Sirius Red staining. This suggested a low steady state level of Col1 mRNA in glomerular cells.

Aged SD (Normal) Rats

Samples of aged SD rats showed increased accumulation of collagen in glomeruli and interstitial space and increased expression of the marker genes. Significantly, the intensity of fibrotic change varied among samples so that one of four samples studied displayed very few changes compared with young animals; fibrotic change in another sample was confined to “polar” regions, and two samples showed uniform accumulation of collagen and elevated expression of marker genes throughout the sections.

Goto Kakizaki (GK)/Wistar (Normal) 48-Week-Old Rats

Samples of both GK and Wistar 48-week-old rats showed an accumulation of collagen in glomeruli and interstitial space. This accumulation was more pronounced in the GK samples. Two samples were used for mRNA isolation: C9 and GK9. Both were hybridized to the probe specific for IGFBP4. The in situ hybridization results showed that the GK sample demonstrated elevated expression of this gene.

Permanent UUO

A known model for fibrosis was employed—unilateral urether occlusion (UUO). One of the urethers was occluded (see below) and animals were sacrifized 1,5,10,15,20 and 25 days following occlusion.

Permanent UUO resulted in rapid activation (5 days of UUO) of collagen synthesis by interstitial cells in both medulla and cortex. By 20-25 days of UUO, significant amounts of interstitial collagen were deposited in the interstitial space while glomerular accumulation of collagen was confined to the outer capsule. Thus, permanent UUO samples provided an acute model of tubulointerstitial renal fibrosis without prominent glomerulosclerotic changes.

The above models can be used as model systems for testing the therapeutic efficacy of modulators of ENDO180 identified via any of the screening systems described.

### Example 5

### Protocol for Permanent Unilateral Ureteral Obstruction (UUO)

Test System

Strain: Male Sprague-Dawley rats (9 weeks of age)

Group Size: n=5 for operated rat; n=3 for sham-operated rats

Number of groups: 6 for both sham-operated and operated (i.e., 1 day, 5 days, 10 days, 15 days, 20 days and 25 days post-operation or post-sham operation)

Procedure

Rats were anaesthetized with Ketamin/Xylazine and the abdominal cavity was opened. After being exposed, the ureter from the right kidney was ligated with a suture over it (UUO). In sham-operated rats, the ureter was exposed but not ligated.

Study Termination

The study was terminated 24 hr, 5 days, 10 days, 15 days, 20 days and 25 days after the UUO procedure or after the sham operation. At this time point, the rats were sacrificed by exsanguination under CO₂ asphyxiation in order to collect the right kidney. After the capsule was removed the kidney was cut transversely. Half was fixed in 10% buffered formalin and the other half was immediately transferred to an eppendorf tube and frozen in liquid nitrogen for RNA analysis.

### Example 6

### Endogenous Expression of ENDO180 in Various Normal Rat Tissues (by in situ Hybridization)

The fragment homologous to rat ENDO180 gene was used as the probe for hybridization with normal rat tissue multiblock. The T3 (sense) probe gave no hybridization signal. The T7 (antisense) probe gave hybridization signal associated with lymphoid cells, tissue macrophages, stromal (fibroblast) cells and some endothelial cells of connective tissue and seminiferous epithelial cells. Significantly, positive cells of each type are not abundant.

Single positive lymphocytes are scattered throughout sections of spleen, thymus and lymph nodes without any specific localization. Positive macrophages can be seen within lamina propria of intestine (duodenum, ileum and colon).

Positive fibroblasts, perivascular and some endothelial cells can be seen in interstitial tissue of salivary glands and in adventitia of trachea. Peculiar pattern of expression was found in testis. Expression was revealed in some (not all) tubular profiles and was confined to basal layer of seminiferous epithelium and fibroblasts within sheaths of fibrous tissue surrounding same profiles.

Thus, results of in situ hybridization study of the ENDO180 expression suggest a rather low level of expression in normal rat tissues.

### Example 7

### Endogenous Expression of ENDO180 in Rat Kidney Samples (by in situ Hybridization)

Material Used

Ribo-probes complementary to rat homologues of the kidney fibrosis candidate genes were hybridized to sections of rat kidney samples. The following samples were included in this study:
1. Permanent UUO multiblock containing sham operated control sample—fixed at 25 days after operation and five UUO samples—fixed at 1, 5, 10, 20 and 25 days after the occlusion.
2. Aged diabetic multiblock containing one sample of 9 month old ZDF kidney and one sample of 12 month old Zucker fa/fa kidney. Both these samples are highly fibrotic. The ZDF sample shows also strong inflammation.
3. Young Zucker fa/fa multiblock containing 2 samples of 3 month old and 2 samples of 6 month old Zucker fa/fa kidneys.
4. Chronic renal failure sample—2 years 7 month old rat kidney.
5. Normal kidney samples

Results

Hybridization of rat kidney sections with probe specific to this gene (rat ENDO180) revealed single interstitial cells located mainly in medulla and single glomerular cells showing expression of this gene in non fibrotic samples. Fibrotic samples demonstrated multiple interstitial cells showing hybridization signal while glomerular signal was not changed comparing to non-fibrotic samples. These results suggest low constitutive expression of this gene in normal rat kidney and considerable involvement of this gene product into interstitial fibrosis. Thus, rat kidney results comply very well with results obtained with human samples (see next section) and strongly support the role of ENDO80 in fibrosis.

### Example 8

### Endogenous Expression of ENDO180 in Human Kidney Samples (by in situ Hybridization)

The human fibrotic kidney samples used for in situ hybridization analysis are as follows:

Normal Kidney Samples
1. Postmortem material—28 years old male. Necropsy performed within 2 hr after the accidental death.
2. Postmortem material—26 years old male. Necropsy performed within 2 hr after the accidental death.
3. Surgical material—34 years old male. Traumatized kidney removed after an accident.

Fibrodc Kidney Samples
1. Sample 11957—65 year old female. Diabetes—for the last 15 years (up to 11 mmol/l glucose), pyelonephritis. Kidney removed by surgery to avoid sepsis.
2. Sample 44561—39 year old female. Diabetes (up to 11.2 mmol/l glucose), pyelonephritis. Kidney removed by surgery to avoid sepsis.
3. Sample C86—63 year old female, diabetes (up to 13 mmol/l glucose). Died from thromboembolism at hospital after amputation of the lower extremity. Necropsy performed within 2 hr after death.
4. Sample 15144—79 year old female. Diabetes (up to 14.8 mmol/l glucose), pyelonephritis. Kidney removed by surgery to avoid sepsis.
5. Sample 39159—66 year old male. Diabetes (up to 17.6 mmol/l glucose), kidney removed by surgery due to kidney stone disease.
6. Sample 4533—32 year old male. Glucose up to 7.2 mmol/l. Kidney removed by surgery due to kidney stone disease.
7. Sample C6—62 year old male. Diabetes (no glucose data available). Patient died from lymphoma Necropsy performed within 2 hr after the death.

Results

In human normal kidney, the expression is confined to glomeruli (signal varying from weak to none) and to stromal cells in renal pelvis. In pathological samples, the expression is in interstitial infiltrating (macrophages), and endothelial cells (rare) as well as in atrophic tubules, and in vSMC (rare).

### Example 9

### In vitro Functional Validation

ENDO180 cDNA containing the complete ORF subcloned into pIRES (pIRESpuro empty vector served as negative control) was transfected into Rat1 and NRK52Ecells. Two independent Rat1 control polyclonal cell populations and two independent Rat1 ENDO180 expressing cell populations were established. Expression of exogenous ENDO180 was verified by Northern blot analysis. ENDO180 over-expressing Rat1 cell pools were next evaluated for collagen deposition and growth rate in the presence of TGF-beta, according to the following protocols:

For cell count, cells were seeded in 96-well plate (2×10³/well) in 200 μl of DMEM with 10% FBS. After overnight culture, the medium was replaced with DMEM containing 2% FBS. 48 hr, 72 hr, 96 hr, or 7 days later the cells were stained with neutral red and the optical density was measured at 540 nm.

For collagen synthesis, cells were seeded in 24-well plate (1×10⁴/well) and grown in 1 ml of DMEM with 10% FBS until confluence (for 96 hr) followed by 72 hr of TGF-β stimulation. (0.2 ng; 0.5 ng; 1 ng; 2 ng/ml TGF-β in the presence of 0.1% BSA). Collagen synthesis was evaluated with a Sirius Red-based colorimetric microassay. Optical density was measured at 540 nm

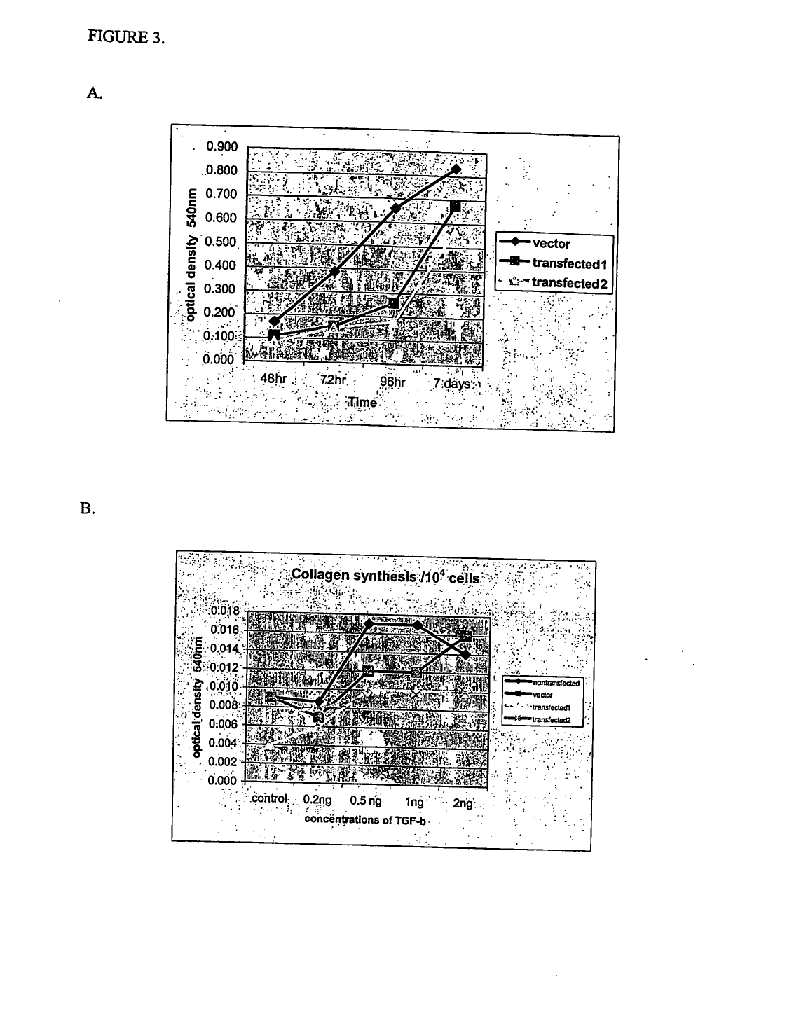

The results are presented in FIGS. 3A and B. It is evident that ENDO180 expression significantly reduces cell proliferation rate and slightly reduces collagen production.

1-54. (canceled)

55. A method comprising contacting a ENDO180 polypeptide receptor on the surface of a cell with an antibody to such polypeptide receptor having conjugated thereto a moiety so as to effect internalization of the antibody within the cell.

56. A method for the treatment of a subject in need of treatment for a disease comprising administering to said subject an amount of an antibody to ENDO-180 polypeptide receptor on the surface of the cell which antibody is internalized by the cell so as to thereby treat the subject.

57. A method of claim 56 wherein the antibody has a moiety conjugated thereto.

58. A method of claim 57 wherein the antibody and moiety are internalized.

59. A method for the treatment of a subject in need of treatment for a disease wherein the disease is fibrosis associated pathology, cancer, osteoarthritis or osteoporosis comprising administering to said subject an amount of an antagonist to ENDO-180 polypeptide in a therapeutically effective amount so as to thereby treat the subject.

60. The method of claim 59 wherein the disease is fibrosis associated pathology and is selected from kidney, liver, lung and heart fibrosis, nephropathy, in particular diabetic nephropathy, chronic renal insufficiency, chronic renal failure and glomerulosclerosis.

61. The method of claim 59 wherein the antagonist is an antibody to ENDO-180 polypeptide.

62. The method of claim 59 wherein the antagonist is an siRNA corresponding to ENDO-180.

63. The method of claim 59 wherein the antagonist is an antisense oligonucleotide corresponding to ENDO-180.
